(19) United States
(12) Patent Application Publication
(10) Pub. No.: US 2011/0262449 A1
(43) Pub. Date: Oct. 27, 2011

(54) METHOD FOR THE TREATMENT OF GOUT OR PSEUDOGOUT

(75) Inventors: Virginie PETRILLI, Lausanne (CH); Fabio Martinon, Lausanne (CH); Jurg Tschopp, Epalinges (CH); Thibault De Smedt, Lutry (CH)

(73) Assignee: TOPOTARGET SWITZERLAND SA, LAUSANNE (CH)

(21) Appl. No.: 13/175,266

(22) Filed: Jul. 1, 2011

Related U.S. Application Data
(63) Continuation of application No. 12/159,842, filed on Jul. 1, 2008, now abandoned.

(30) Foreign Application Priority Data
Jan. 6, 2006 (EP) PCT/EP2006/050060

(51) Int. Cl. A61K 39/395; A61P 19/06; A61P 29/00; A61P 17/00; A61P 19/02; A61P 17/06; A61P 19/04; A61P 11/06; A61P 25/00; A61P 11/00; A61P 1/00; A61P 9/10; A61P 25/28; A61P 25/16; C07D 225/06; C07K 16/24; A61K 31/33; A61K 38/17

(52) U.S. Cl. 424/145.1; 424/158.1; 514/16.7; 540/461; 530/389.2; 530/388.23; 514/183

(57) ABSTRACT

The present invention relates to a new method, and the process to manufacture a medicament, for treating gout or pseudogout, comprising administering an effective amount of inhibitors blocking IL-1 or its maturation by the NALP3 inflammasome.

This application is a Continuation of U.S. application Ser. No. 12/159,842, filed Jul. 1, 2008, which is a National Stage Application of European Application No. PCT/EP2007/050143, filed Jan. 8, 2007, which claims priority to European Application PCT/EP2006/050060, filed Jan. 6, 2006, which claims priority to U.S. application Ser. No. 11/539,851, filed Oct. 9, 2006, the entire contents of which are hereby incorporated by reference in their entirety.

The present invention relates to a new method, and to the process of manufacturing a medicament, for the treatment of gout or pseudogout, comprising administering an effective amount of inhibitors blocking IL-1 or its maturation by the NALP3 inflammasome.

### BACKGROUND OF THE INVENTION

Development of the acute and chronic inflammatory responses, known as gout and pseudogout, is associated with the deposition of monosodium urate (MSU) or calcium pyrophosphate dihydrate (CPPD) crystals, respectively, in joints and periarticular tissues. Although MSU crystals were first identified as the etiologic agent of gout in the 18th century¹, and more recently as a “danger signal” released from dying cells², little is known on the molecular mechanisms underlying MSU- or CPPD-induced inflammation.

Few therapies are available today for the treatment of gout or pseudogout, for patients suffering from the symptoms of gout or pseudogout. These treatments generally use colchicine and other compounds. However, such treatments are not satisfactory. As an example, use of oral colchicine is limited by side effects such as nausea and abdominal pain. Information regarding usual gout treatments and side effects is given by Cannella & Mikuls (The American Journal of Managed Care, November 2005, Vol. 11, NO. 15, SUP., S541-S458) and on the Web site http://www.arthritis.ca/types%20of%20arthritis/gout/default.asp?s=1.

Gout belongs to a group of pathologies collectively named “autoinflammatory diseases”. They comprise a heterogeneous group of pathologies characterized by spontaneous periodic inflammation and fever in the absence of infectious or autoimmune causes³. Hereditary periodic fevers (HPFs), Muckle-Wells syndrome, familial mediterranean fever (FMF), familial cold-induced autoinflammatory syndrome (FCAS), and the metabolic disorders gout and pseudogout are examples of such inflammatory maladies. There is a difference between gout and autoimmune diseases like rheumatoid arthritis, osteoarthritis or systemic-onset juvenile idiopathic arthritis (SoJIA). In fact, it is accepted that autoimmune diseases comprise an adaptative immune component (B cells and T cells), which is responsible for the recognition of self-components by re-arranged, clonally selected receptors (antibodies and T cell receptors). It could be initiated by infection, resulting in cross-reaction with antigens of the self. Alternatively, a genetic predisposing background could lead to these pathologies. By contrast, gout only involves innate immune system components, in particular the NOD-like receptors, and not adaptative immune components. Gout does not appear to be initiated by pathogens. Rather, it arises from a metabolic malfunction, the augmentation of MSU crystals in some regions of the body (Choi, H K et al. 2005. Pathogenesis of gout. Ann. Intern. Med. 143:499).

IL-1β, known as the endogenous pyrogen, is a highly inflammatory cytokine whose production is tightly controlled at least at three distinct steps⁹.

The first step involves the production of the proIL-1β protein (p35). The second step involves the cleavage of the precursor proIL-1β to produce the active IL-1β protein (p17). This reaction relies on the activation of a caspase-1 activating complex, the best characterized being the inflammasome^{10, 11}. In the third step, IL-1β is released into the extracellular environment

Upon activation, the inflammasome is formed by a member of the NOD-LRR (nucleotide-binding oligomerization domain-leucine-rich repeat) protein family such as NALP1, NALP2, NALP3/Cryopyrin or Ipaf, and the adaptor protein, ASC, that connects the NOD-LRR proteins with caspase-1¹². Signals and mechanisms leading to inflammasome activation are still poorly understood. Muramyl dipeptide (MDP), a degradation product of the bacterial cell wall component peptidoglycans, and a contaminant of crude LPS, was recently shown to activate a NALP3 inflammasome¹³ through NALP3's LRR domain, suggesting that NALPs, like Toll-like receptors (TLRs), are fundamental for microbial detection¹⁴. However, the inflammasome is also proficient in sensing stress or endogenous “danger signals”, such as extracellular ATP or hypotonic stress^{10, 11, 15}. Recently, MSU crystals were identified as a “danger signal” formed following release of uric acid from dying cells².

Inventors have shown that MSU and CPPD engage the NALP3 inflammasome, resulting in the production of active IL-1α, IL-1β and IL-18.

Macrophages from mice deficient in various components of the inflammasome, such as caspase-1, ASC and NALP3, are defective in crystal induced IL-1 activation, and ensuing neutrophil influx. Likewise, IL-1 receptor type I (IL-1RI) deficiency results in impaired neutrophil recruitment in MSU- or CPPD-induced inflammation. In the same line of evidence, administration of blocking monoclonal antibodies to IL-1 receptor type I or anakinra (Kineret, IL-1Ra), but not blocking monoclonal antibodies to TNF, abrogates MSU-induced neutrophil recruitment in mice. Likewise, inhibitors of the HSP90 chaperone inhibit the formation of the NALP3 inflammasone. These inhibitors also block IL-1 secretion by human monocytes and mouse macrophages, and inhibit MSU-induced neutrophil recruitment in mice.

These findings provide insight into the molecular processes underlying the inflammatory conditions of gout and pseudogout, and identify new drugs and medicaments suitable for the treatment of such conditions.

### BRIEF DESCRIPTION OF THE INVENTION

The present invention relates to a new method for the treatment of gout or pseudogout, comprising administering an effective amount of inhibitors blocking IL-1 or its maturation by the NALP3 inflammasome.

NALP3 inflammasome is a conjugate of caspase-1, ASC and NALP3.

According to the invention, inhibitors blocking IL-1 maturation by the NALP3 inflammasome comprises inhibitors of the formation of NALP3 inflammasome and/or inhibitors of the activity of NALP3 inflammasome.

Inhibitors of the formation of NALP3 inflammasome are particularly inhibitors of expression and/or activity of caspase-1, and/or ASC and/or NALP3.

Moreover, we have found that assembly of the NALP3 inflammasome necessitates the transient association of NALP3 to the heat shock protein HSP90 and to the client protein Sgt1. Therefore, inhibitors of the formation of NALP3 inflammasome are also comprised of inhibitors of expression and/or activity of HSP90 and/or Sgt1, more particularly inhibitors of HSP90.

According to the invention, inhibitors of activity of NALP3 inflammasome may be inhibiting processing of proIL-1β into IL-1β by NALP3 inflammasome.

Inhibitors blocking IL-1 are preferably inhibitors blocking IL-1α and/or IL-1β.

These inhibitors are preferably selected among inhibitors of the activity of IL-1, inhibitors of the IL-1 receptor and IL-1 receptor antagonists.

### DETAILED DESCRIPTION OF THE INVENTION

Inhibitors blocking IL-1 or its maturation by the NALP3 inflammasome are composition of matters such as small molecules, DNA or RNA sequences, proteins, antibodies, which action inhibit formation of IL-1, and/or activity of the same IL-1, and/or maturation of IL-1 by the NALP3 inflammasome.

Small molecular weight compounds are chemical compounds of a molecular weight below about 500 Da. These chemical compounds are from natural origin, modified natural compounds or fully synthetic compounds. Natural compounds may be obtained by extraction of biological materials such as plants, plant extracts or any other medium comprising biological materials. They can be also obtained by complete or partial synthesis. They can be used as such or in a saline form with known pharmaceutically acceptable salts.

For example, small molecular weight compounds fitting in the ATP binding site of NALP3, may be used as an inhibiting agent of the NALP3 inflammasome. Sequence analysis of NALP3 revealed the presence of a putative ATP binding site. Small molecular weight compounds inhibitors have been successfully developed for other proteins with related ATP biding sites (i.e. kinases, phosphatases, and phosphodiesterases), indicating that one can reasonably anticipate identifying such inhibitors for NALP3.

Small molecular weight compounds specifically blocking HSP90, such as geldanamycin, or less toxic derivates, such as 17-AAG (17-(Allylamino)-17-demethoxygeldanamycin) or 17-DMAG (17-(Dimethylaminoethylamino)-17-demethoxygeldanamycin) and their pharmaceutically acceptable salts, have been successfully developed. These compounds are being evaluated for the treatment of cancers. These inhibitors are also effective in the HSP90-dependent assembly of the NALP3 inflammasome, and may be used in an effective amount to manufacture a medicament for treating gout or pseudogout.

Other geldanamycin derivatives are known in the art such as compounds disclosed in U.S. Pat. No. 4,261,989, US 2004-0235813, WO 02/36574, WO 02/079167, WO 03/02671 and WO 2005/095347, which content is incorporated herein by reference.

Other inhibitors of HSP90 are also disclosed in the art, such as compounds disclosed in WO 2006/095783, WO 2006/092202, WO 2006/090094, WO 2006/087077, WO 2006/084030, WO 2005/028434, WO 2004/072051, WO 2006/079789, US 2006-0167070, WO 2006/075095, US 2006-0148817, WO 2006/057396, WO 2006/055760, WO 02/069900, WO 2006/052795, WO 2006/050373, WO 2006/051808, WO 2006/039977, US 2006-0073151, EP 1 642 880, EP 1 631 267, EP 1 628 667, US 2006-0035837, WO 2006/008503, WO 2006/010595, WO 2006/010594, WO 2006/003384, WO 2005/115431, EP 1 620 090, WO 2005/061461, WO 2005/063222, US 2005-0049263, WO 2004/050087, WO 2004/024142, WO 2004/024141, WO 03/067262, WO 03/055860, WO 03/041643, WO 03/037860, which content is incorporated herein by reference.

Interleukin-1 inhibitors are known in the art, such as inhibitors disclosed in WO 89/11540, or in many scientific articles, such as Nishihara & al (Infect Immun 1988 November; 56(11): 2801-2807) or Peled & al (blood, Volume 79, Issue 5, pp. 1172-1177). Other interleukin antagonists are known in the art, such as small molecules disclosed in U.S. Pat. No. 6,417,202. The content of the above publications is incorporated herein by reference.

According to the invention, inhibitors of IL-1 are preferably selected among the group consisting of inhibiting agents blocking IL-1α and inhibiting agents blocking IL-1β.

In a preferred embodiment, the inhibitor of IL-1 is an IL-1 receptor type I (IL-1RI) antagonist, natural or synthetic, particularly IL-1Ra also known as anakinra, marketed under the name Kineret®.

In another preferred embodiment, inhibitors of IL-1 are selected among antibodies inhibiting activity of IL-1α and/or IL-1β. Such anti-IL-1α and/or β antibodies are polyclonal or monoclonal antibodies, preferably monoclonal antibodies.

In another preferred embodiment, inhibitors of IL-1 are selected among antibodies preventing binding of IL-1α or IL-1β to its receptor. Such anti-IL-1RI antibodies are polyclonal or monoclonal antibodies, preferably monoclonal antibodies.

Such antibodies are known in the art, some being disclosed for their use in therapy for the treatment of other diseases, such as rheumatoid arthritis and osteoarthritis (see for instance WO 03/073982, enclosed herein by reference).

Moreover, the person skilled in the art is able to identify and prepare new antibodies using standard technologies.

In preferred embodiments, antibodies are humanized antibody, i.e. antibodies that are composed partially or fully of amino acid sequences derived from a human antibody germline or a rearranged sequence and made by altering the sequence of an antibody having non-human complementarity determining regions (CDR).

The framework regions of the variable regions are substituted by corresponding human framework regions leaving the non-human CDR substantially intact. The framework region may be entirely human or may contain substitutions in regions that influence binding of the antibody to the target antigen. These regions may be substituted with the corresponding non-human amino acids.

Humanized antibodies have several potential advantages for use in human therapy more particularly regarding non-recognition by the human immune system and a longer half-life in the circulation than non-human antibodies.

Inhibitors blocking IL-1 or its maturation by the NALP3 inflammasome are administered following standard procedures and using standard pharmaceutical compositions.

Inhibitors blocking IL-1 or its maturation by the NALP3 inflammasome are administered using standard administration techniques, preferably peripherally by injection or infusion, intravenous, intraperitoneal, intramuscular or subcutaneous, but also by other routes such as pulmonary, intranasal, buccal, sublingual, transdermal, oral, or suppository administration.

Pharmaceutical compositions for Inhibitors blocking IL-1 or its maturation by the NALP3 inflammasome are known in the art, and are designed to be appropriate for the selected mode of administration. Pharmaceutically acceptable carriers, excipients as well as buffers, surfactants, preservatives, solubilizing agents, stabilizing agents are used according to the known practice.

In preferred embodiments, preferred IL-1Ra or anti-IL-1α and β or anti-IL-1RI antibodies are administered once a day, preferably once a week, even more preferably once a month in a dose sufficient to inhibit IL-1α and β activity.

The person skilled in the art, and more particularly the physician ordering treatment of gout or pseudogout is able to determine the said dose taking into consideration inter alia the development stage of the disease, the age, weight and general condition of the patient.

Recommended dose of anti-IL-1α and β antibodies is comprised between 1 and 20 mg/kg, preferably between 2 and 10 mg/kg, more preferably from 3 to 5 mg/kg. Preferred route of administration is intravenous infusion.

Infusions can be given on specific administration programs determined by the physician. Such program may comprise additional infusions at 1 or 2 and 5 or 6 weeks after the first infusion, followed eventually by further infusions every 8 to 10 weeks thereafter.

The recommended dose of IL-1Ra is comprised between 50 and 150 mg/day, administered daily. Preferred route of administration is subcutaneous injection.

In the method according to the invention, NALP3 inflammasome inhibiting agents may be used combined with other therapeutic agents such as known anti-gout compounds or compositions and known anti-inflammatory compounds or compositions.

Known anti-gout compounds and compositions are selected among colchicines, or compositions preventing accumulation of uric acid such as allopurinol or uricase.

Known anti-inflammatory compounds and compositions are selected among corticoids, such as prednisone, betamethasone, dexamethasone, methylprednisolone, prednisolone, cortivazol, hydrocortisone, triamcinolone, and non steroids such as indometacine, sulindac, tiaprofenic acid, alminoprofene, diclofenac, etodolac, flurbiprofene, ibuprofene, ketoprofene, nabumetone, naproxene, meloxicam, piroxicam, tenoxicam, celecoxib, refecoxib and any other anti-inflammatory compound listed in the pharmacopea.

The present invention also relates to the use of inhibitors blocking IL-1 or its maturation by the NALP3 inflammasome for the preparation of a medicament used in the treatment of gout or pseudogout as disclosed above.

The present invention also concerns the use of HSP90 inhibitors, as disclosed above, including 17-AAG or 17-DMAG, for the treatment of inflammatory disorders in a mammal, particularly for the treatment of inflammatory disorders associated with the formation of an inflammasome, more particularly disorders associated with NALP3 inflammasome formation.

It also concerns the use of HSP90 inhibitors, as disclosed above, including 17-AAG or 17-DMAG, for the preparation of a medicament used in treatment of inflammatory disorders in a mammal, particularly for the treatment of inflammatory disorders associated with the formation of an inflammasome, more particularly disorders associated with NALP3 inflammasome formation.

Such inflammatory disorders are more particularly selected among autoimmune and auto-inflammatory diseases, such as gout, pseudogout, Muckle-Wells Syndrome, hereditary periodic fevers, Familial Mediterranean Fever, Familial Cold-induced Autoinflammatory Syndrome, Blau Syndrome, rheumatoid arthritis, osteoarthritis, systemic-onset juvenile idiopathic arthritis, psoriasis, lupus, multiple sclerosis, asthma, chronic obstructive pulmonary disorder, inflammatory bowel disease, Crohn's disease, atherosclerosis, Alzheimer's disease, Parkinson's disease.

Such inhibitor of HSP90 may indeed be used alone or in combination with other anti-inflammatory compounds and compositions disclosed above.

### DESCRIPTION OF THE FIGURES

FIG. 1: Monosodium urate crystals (MSU) and calcium pyrophosphate dihydrate (CPPD) activate IL-1β cleavage and release.

a-c, THP1 cells were stimulated for 6 h with the indicated amounts/ml of MSU crystals (a), CPPD (b) or with 50 μg/ml of pure LPS, MSU, allopurinol crystals, CPPD crystals, diamond crystals, aluminum particles, zymosan, crude preparations of LPS, or 5 mM of extracellular ATP as indicated (c). Supernatants (SN) were analyzed for the presence of mature IL-1β, IL-18 or caspase-1, and cell extracts (Cell) for the presence of proIL-1β and proIL-18. d, human monocytes were stimulated with 50 μg/ml of the indicated crystals for 6 h and analyzed by Western blot for IL-1β activation or by ELISA for released caspase-1 and IL-1β.

FIG. 2: The NALP3 inflammasome is required for the maturation of IL-1α and β.

Mouse macrophages from Wild-Type (+/+), caspase-1 (Casp1) or MyD88 deficient mice (a), ASC deficient mice or littermate controls (b), and NALP3 deficient mice or littermate controls (c) were stimulated as indicated in the presence of ultra pure LPS (1 μg/ml, Alexis or Invivogen) in order to induce the synthesis of precursor proIL-1β. In (c), ultra pure LPS was added 1 h before stimulation. Supernatant (SN) or cell extracts (Cell) were analyzed by Western blot as indicated. (d) Macrophages from WT or NALP3 KO were primed with LPS and then activated with MSU crystals. SN were tested for IL1 and TNF content by ELISA.

FIG. 3: Gene targeting strategy for disruption of the mouse NALP3 gene.

a, an EGFP cassette was inserted in frame with the ATG of exon 2. The EGFP cassette is followed by the SV40 poly(A) tail, resulting in the disruption of the NALP3 gene. A selection cassette PGK-neo flanked by 2 loxP sites was inserted in the intron 2. The neo cassette was deleted by backcrossing the mice with a Cre-expressing deletor strain (C57BL/6). b, PCR genotyping of KO, WT and heterozygote mice.

FIG. 4: Monosodium urate crystals (MSU)-mediated activation of IL-1β occurs independently of the ATP-receptor P2X₇.

THP1 cells were pre-treated with the P2X antagonist pyridoxal-phosphate-6-azophenyl-2′,4′-disulfonic acid (PPADS, Alexis) for 30 min and subsequently stimulated for 6 h with MSU crystals (50 μg/ml). Supernatants (SN) were analyzed for the presence of mature IL-1β and cell extracts (Cell) for the presence of proIL-1β.

FIG. 5: IL-1β maturation is an early event following MSU and CPPD stimulation.

a, THP1 cells were stimulated with MSU, CPPD or Zymosan (Zym) for the indicated times in the presence or absence of the caspase-1 inhibitor zYVAD-fmk. Supernatants were analyzed for TNF-α (gray bars) and IL-1β (black bars) production by ELISA. b, human monocytes were incubated with MSU or CPPD in the presence of two concentrations of IL-1Ra. TNF-α and IL-6 production was monitored by ELISA.

FIG. 6: IL-1β maturation is blocked by colchicine.

THP1 cells were stimulated with MSU, CPPD or ATP in the presence or absence of colchicine (colch). Maturation of IL-1β was analyzed by Western Blot.

FIG. 7: IL-1β maturation in primary macrophages is blocked by geldanamycin

Human primary monocytes were treated overnight with 200 nM geldanamycin (GA), then stimulated for 6 h with MSU (100 μg/ml), PGN (50 μg/ml) or 5 mM (ATP). Supernatants were collected and assessed for IL-1β processing.

FIG. 8: Effect of 17-AAG and 17-DMAG on IL-1β production by monosodium urate crystal-stimulated mouse peritoneal exudate cells and human blood monocytes.

BALB/C mice were intraperitoneally injected with a 4% solution of thioglycollate. Six days later, peritoneal exudate cells (PEC) were collected by washing the peritoneal cavity with phosphate buffer saline. Cells were distributed in wells of tissue culture 12 well plates (10⁶ cells/well). Non-adherent cells were washed and adherent cells were treated with 1 μg/ml of Ultrapure LPS (Invivogen) in the presence or the absence of 200 nM of 17-AAG (Invivogen). (a) After overnight culture, the supernatants were collected and used for determination of TNF by ELISA. (b, c) Fresh medium was added on the cells in the presence or the absence of 2 mM of ATP or 100 μg/ml MSU. After 5 hours, the supernatants were collected and IL-1β was determined by ELISA. (d) Mouse peritoneal macrophages were treated with 500 ng/ml of Ultrapure LPS (Invivogen) in the presence or the absence of 50 nM of 17-DMAG (Invivogen). 2 hours later, cells were stimulated with 500 μg/ml MSU. After 4 hours, the supernatants were collected and IL-1β was determined by ELISA. (e) Human blood monocytes were treated with 1 μg/ml of Ultrapure LPS (Invivogen) in the presence or the absence of graded doses of 17-DMAG (Invivogen). After overnight culture, cells were stimulated with 100 μg/ml MSU. After 6 hours, the supernatants were collected and IL-1β was determined by ELISA.

FIG. 9: Degradation of NALP3 upon geldanamycin treatment.

Flag-tagged NALP3 T-rex cell were transfected with siRNA against Sgt1 or a scramble siRNA. Forty-eight hours post transfection, NALP3 was induced with doxycyclin. Cell were treated eight hours with geldanamycin 1 μM. The degradation of NALP3 in the cell lysates was analyzed by Western blot.

FIG. 10: Role of the inflammasome in a mouse model of crystal-mediated peritonitis.

(a-c) The indicated wild-type or mutant mice received 0.5 ml i.p. of sterile PBS alone or supplemented with 1 mg of the indicated crystals or 0.2 mg of zymosan. Neutrophil influx was quantified 6 h later (values are ±S.E.M of n=4 to 6 mice per group). Unpaired Student's t-Test was used to calculate p value.

FIG. 11: IL-1R engagement/triggering plays a determining role in gout symptoms in a mouse model.

The indicated wild-type or mutant mice received 0.5 ml i.p. of sterile PBS alone or supplemented with 1 mg of the indicated crystals or 0.2 mg of zymosan. Neutrophil influx was quantified 6 h later (values are ±S.E.M of n=4 to 6 mice per group). Unpaired Student's t-Test was used to calculate p value.

FIG. 12: Effect of anakinra (Kineret) and anti-IL-1 receptor of type 1 (IL-1RI) monoclonal antibody on monosodium urate crystal-induced inflammation in mice.

Aseptic peritonitis was induced in female C57BL/6 mice by injection of 500 μg monosodium urate crystals (MSU) with PBS, 200 μg anakinra (Kineret, Amgen Inc., Thousand Oaks, USA), 200 μg anti-IL-1RI monoclonal antibody (BD Pharmingen, San Jose, USA), 200 μg anti-IL-1α monoclonal antibody (Biolegend, San Diego, USA), 200 μg anti-IL-1β monoclonal antibody ((Biolegend, San Diego, USA), or combination of 200 μg anti-IL-1α monoclonal antibody and 200 μg anti-IL-1β monoclonal antibody. After 6 h, mice were killed and the peritoneal cavity was washed with 10 ml of cold PBS. The lavage fluids were analyzed for neutrophil recruitment by FACS using the neutrophil markers CD11b and Gr1 (Ly-6G). Values are ±s.e.m. of total neutrophil counts with 4-5 animals per group. Unpaired Student's t test was used to calculate P value.

FIG. 13: Absence of inhibition of MSU-induced neutrophil recruitment by anti-TNFα monoclonal antibody.

Aseptic peritonitis was induced in female BALB/C mice by injection of 500 μg monosodium urate crystals (MSU) with PBS, 200 μg anakinra (Kineret, Amgen Inc., Thousand Oaks, USA) or 200 μg anti-TNFα monoclonal antibody (BD Pharmingen, San Jose, USA). After 6 h, mice were killed and the peritoneal cavity was washed with 10 ml of cold PBS. The lavage fluids were analyzed for neutrophil recruitment by FACS using the neutrophil markers CD11b and Gr1 (Ly-6G). Values are ±s.e.m. of total neutrophil counts with 4-5 animals per group. Unpaired Student's t test was used to calculate P value.

FIG. 14: Effect of 17-DMAG, a water-soluble analog of geldanamycin, on monosodium urate crystal-induced inflammation in mice.

Female BALB/C mice were treated intraperitoneally with NaCl 0.9% or 25 mg/kg 17-DMAG. Aseptic peritonitis was induced 1 h later by injection of 500 μg monosodium urate crystals (MSU). After 5 h, mice were killed and the peritoneal cavity was washed with 10 ml of cold PBS. The lavage fluids were analyzed for neutrophil recruitment by FACS using the neutrophil markers CD11b and Gr1 (Ly-6G). Values are ±s.e.m. of total neutrophil counts with 5 animals per group. Unpaired Student's t test was used to calculate P value.

### MATERIAL AND METHODS

Crystal Preparation.

MSU crystals were prepared as described³¹. Briefly 1.68 gm of uric acid in 0.01 M NaOH was heated to 70° C. NaOH was added as required to maintain pH between 7.1 and 7.2 and the solution was filtered and incubated at RT with little stirring slowly and continuously 24 h. CPPD was obtained by mixing a calcium nitrate solution (0.1 M final concentration) with an acidic solution of sodium pyrophosphate (final concentration, 25 mM of Na₂P₂O₇ and 30 mM HNO₃). The milky-white precipitate formed CPPD crystals following filtration and 24 h incubation at 50 to 60° C.³². Allopurinol crystals were generated as described before². Diamond crystals (1-3 microns) were kindly provided by microdiamant AG, Lengwil (Switzerland). All the crystals were kept sterile, washed with ethanol, dried, autoclaved, re-suspended in PBS by sonication and were examined under phase and polarizing microscopy.

Primary Human Monocyte and THP1 Preparation and Stimulation.

THP1 were stimulated for 3 h with 0.5 μM of PMA the day before stimulation as described¹⁰. This treatment increases the phagocytic properties of the cells and induces a constitutive production of proIL-1β³³. Human monocytes were purified as described before³⁰. All cells were stimulated in OptiMEM medium as indicated. Human mature IL-1β was detected with a specific antibody directed against the cleaved epitope (D116) from Cell Signalling, or with an Enzyme-linked immunoabsorbent assay (ELISA) from BD bioscience. TNF and IL-6 were detected by an ELISA from ImmunoTools and caspase-1 by an ELISA from Alexis. IL-18 was detected with an antibody from MBL (D044-3) and Caspase-1 with an antibody from Santa Cruz Biotechnology (sc-622). The antibody against human IL-1β was a gift from Roberto Solari, Glaxo. z-YVAD-fmk was purchased from Alexis Biochemicals. IL-1ra (anakinra, Kineret) was from Amgen (thousand Oaks, USA).

Mice

NALP3 targeting vector (FIG. 5) was electroporated into C57BL/6 ES cells (Ozgene). Homologous recombinant ES cells were identified by Southern blot analysis, and microinjected into C57BL/6 blastocysts. Offsprings were backcrossed to C57BL/6 mice and germline transmission was confirmed by PCR of tail genomic DNA (FIG. 5b). Screening of NALP3 deficient mice by PCR genotyping was carried out using the following primers on tail genomic DNA: 5′GCTCAGGACATACGTCTGGA (forward in intron 1), 5′TGAGGTCCACATCTTCAAGG (reverse in exon2) and 5′TTGTAGTTGCCGTCGTCCTT (reverse in EGFP cassette). RT-PCR analysis of cDNA isolated from NALP3+/+ and NALP3−/− macrophages confirmed the absence of NALP3. ASC deficient mice were a generous gift of Vishva Dixit (Genentech, San Francisco) and were described previously¹¹. Caspase-1-deficient mice (C57BL/6) were a kind gift of Richard Flavell (Yale University, School of Medecine), MyD88-deficient mice (C57BL/6) were obtained from Shizuo Akira (Research Institute of Microbial Diseases, Osaka University), IL-1R (BALB/C)-deficient mice were obtained from Manfred Kopf (ETH, Zurich). Procedures used in this study complied with federal guidelines.

Mouse Macrophage Preparation.

Eight to twelve week-old mice of indicated genotypes were injected i.p. with 4% thioglycollate solution, and macrophages were collected by peritoneal lavage 3-6 days later. Cells were plated at the density of 7×10⁵ cells in twelve-well dishes and non-adherent cells were removed after 3 h. Cells were cultured in RPMI complemented with 10% FCS, sodium pyruvate, penicillin/streptomycin and L-glutamin. All cells were stimulated in OptiMEM medium as described above. Caspase-1 was analyzed using an antibody from Santa Cruz Biotechnology (sc-514) and ASC using an antibody as described previously³⁴. The antibody against mouse IL-1β was a gift from Roberto Solari, Glaxo. The following mouse ELISA kits were used: R&D systems for TNF and IL-1β, and BD biosciences for IL-6.

In Vivo Mouse Peritonitis Model.

Peritonitis was induced by injection of MSU crystals or zymosan in 0.5 ml sterile PBS. After 6 h, mice were euthanized by CO₂ exposure and peritoneal cavities were washed with 10 ml of PBS. The lavage fluids were analysed for PMN recruitment by FACS using the neutrophil marker Ly-6G and CD11b (BD Pharmingen).

### EXAMPLES

The following examples are intended to illustrate particular embodiments and not limit the scope of the invention.

### Example 1

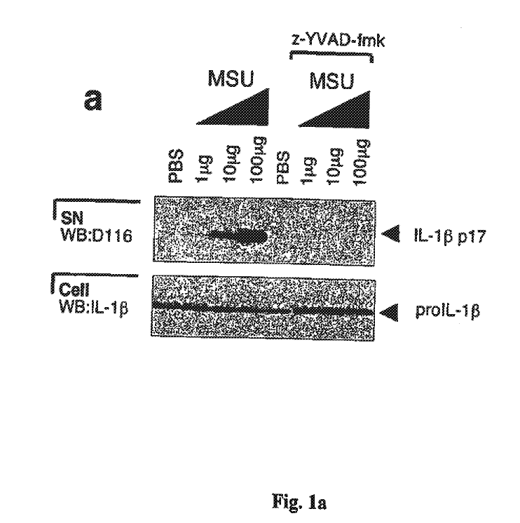

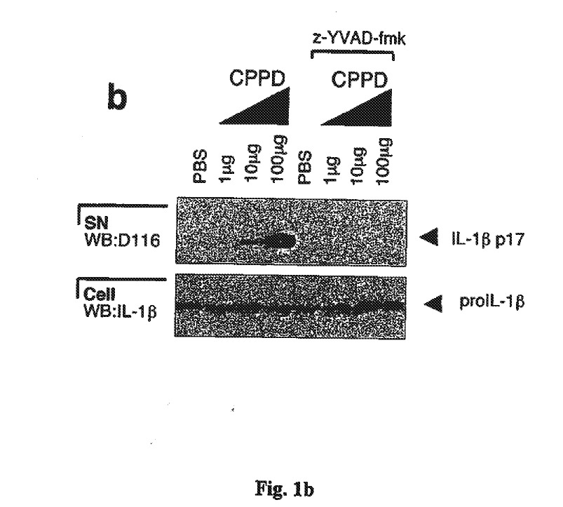

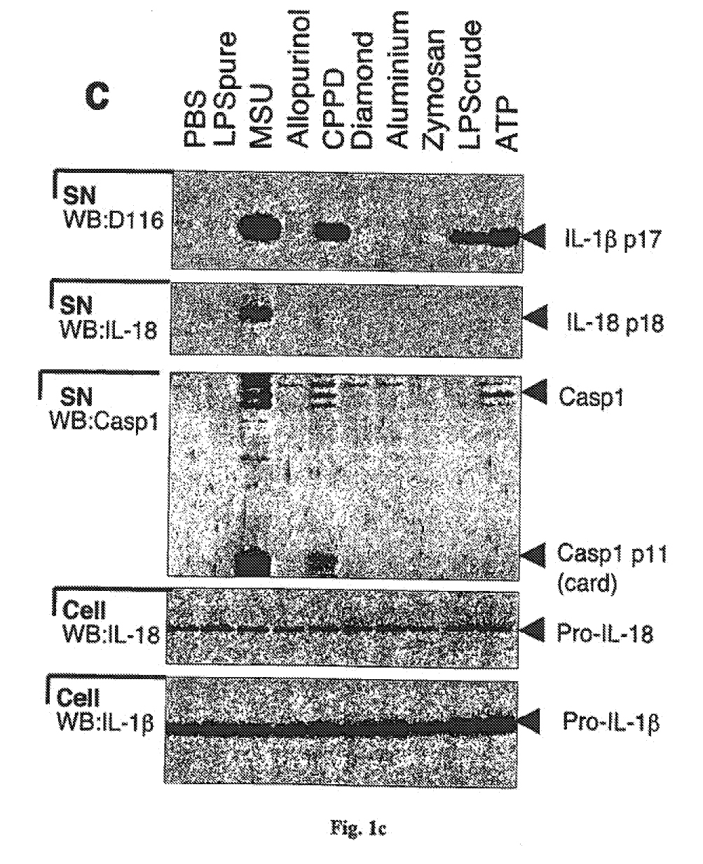

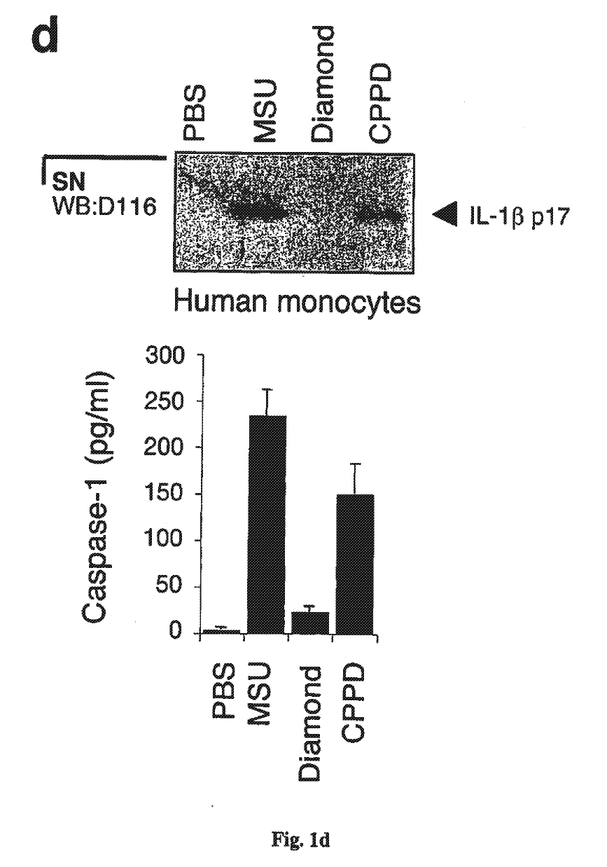

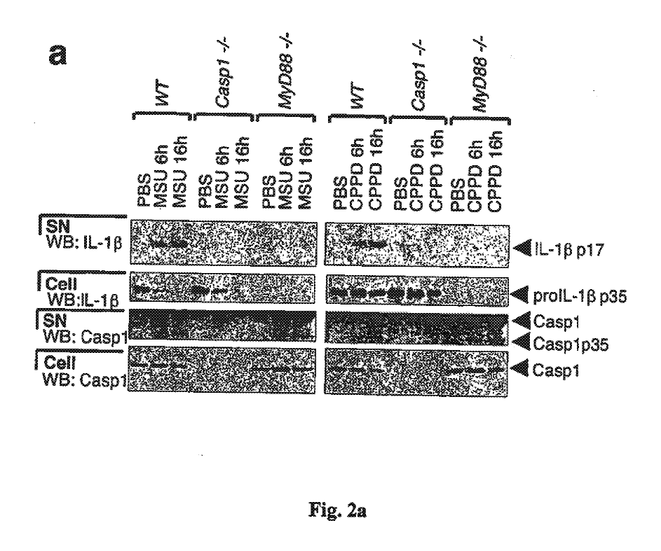

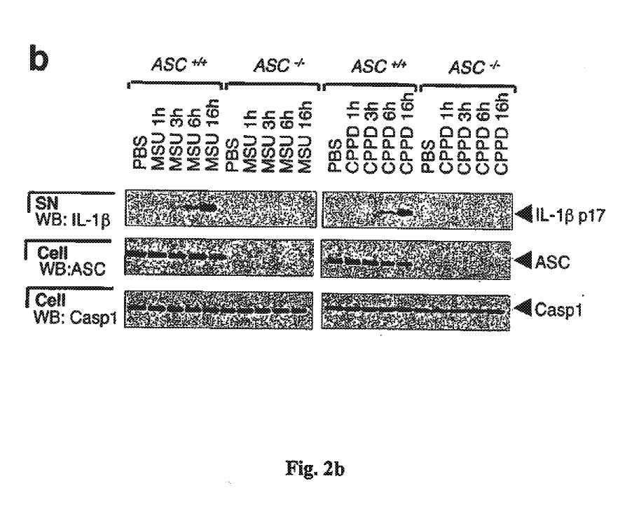

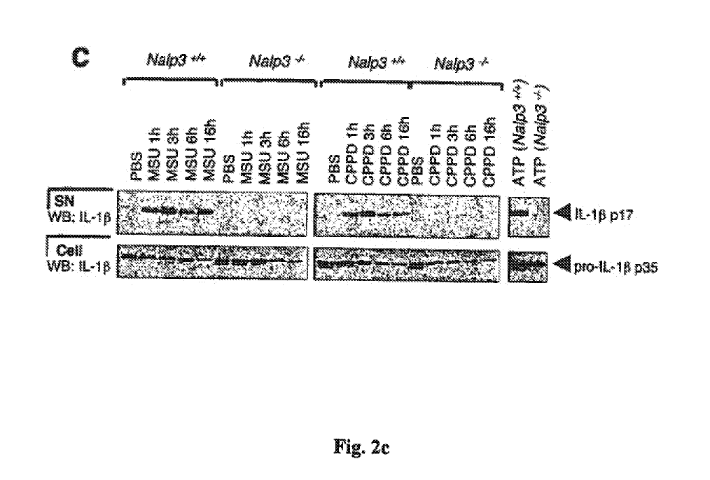

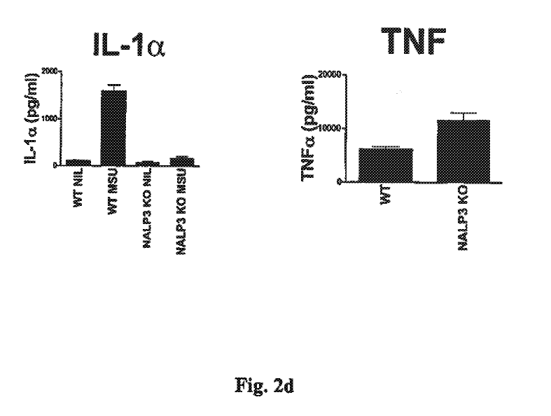

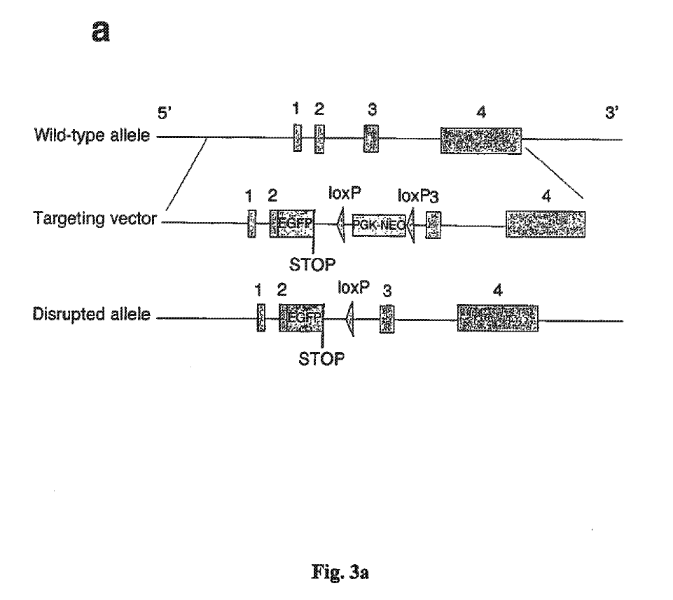

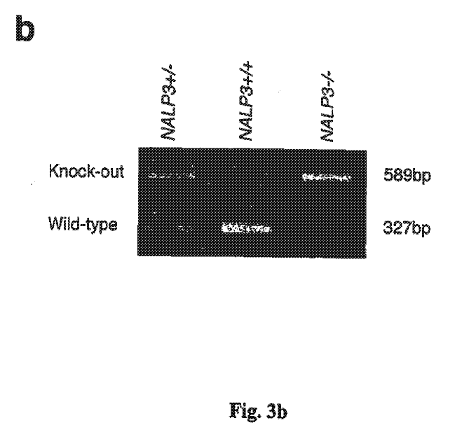

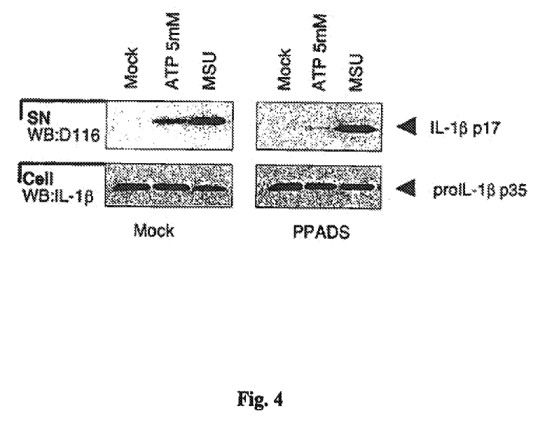

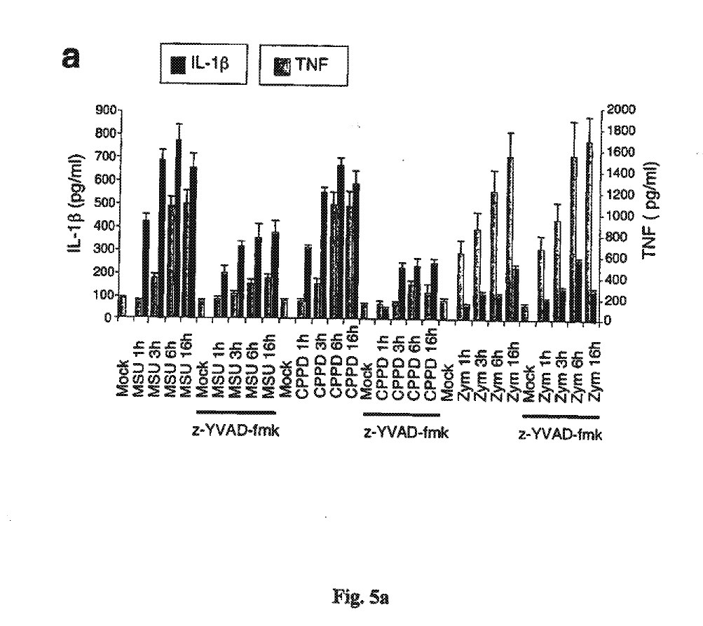

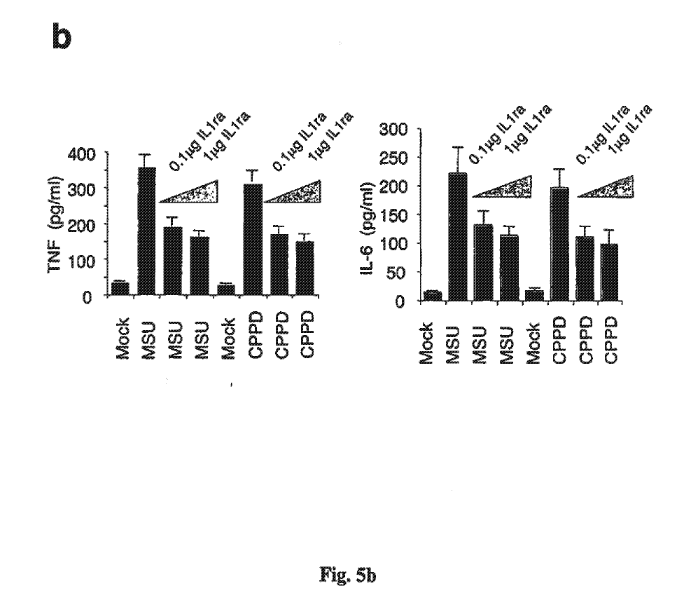

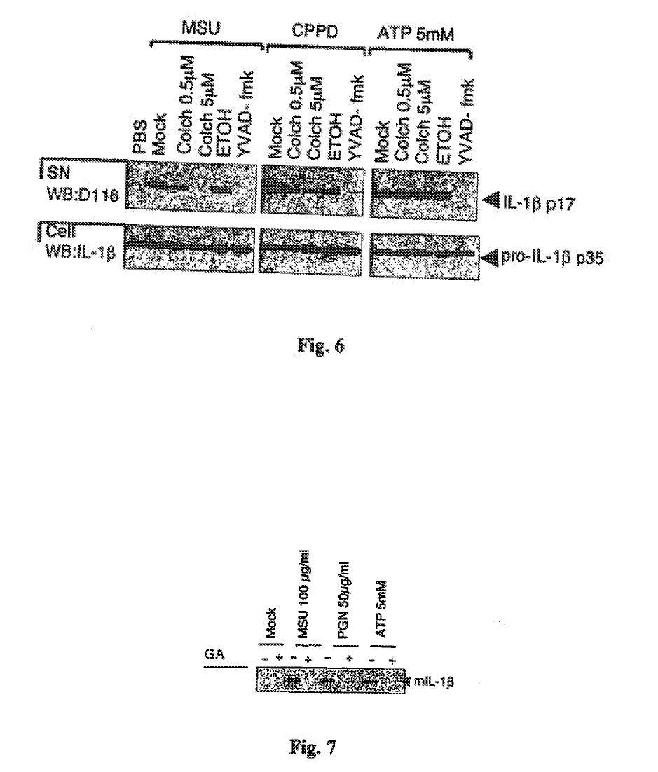

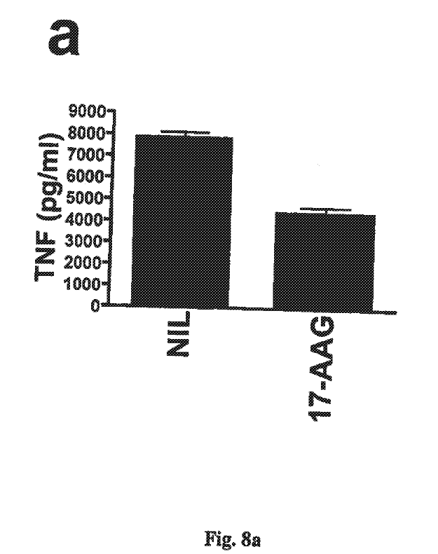

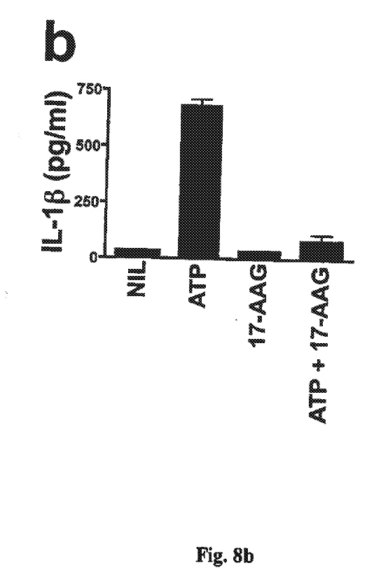

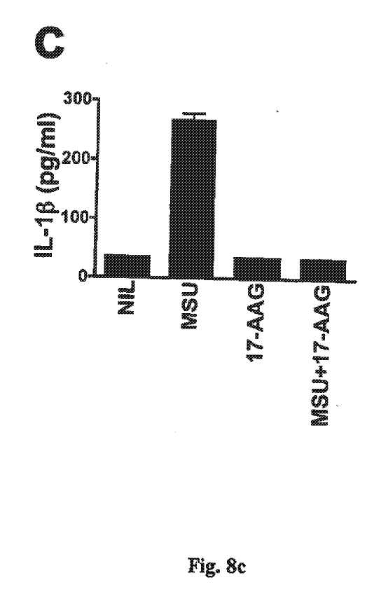

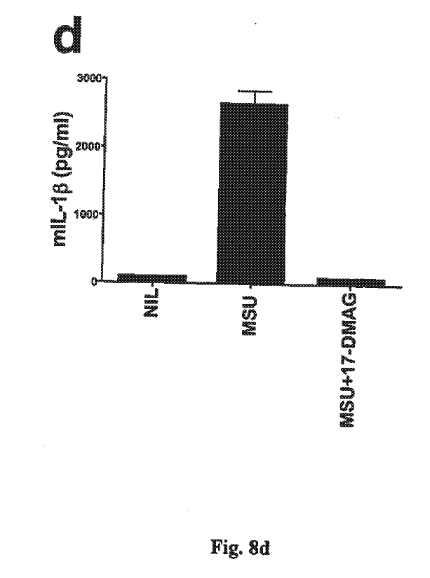

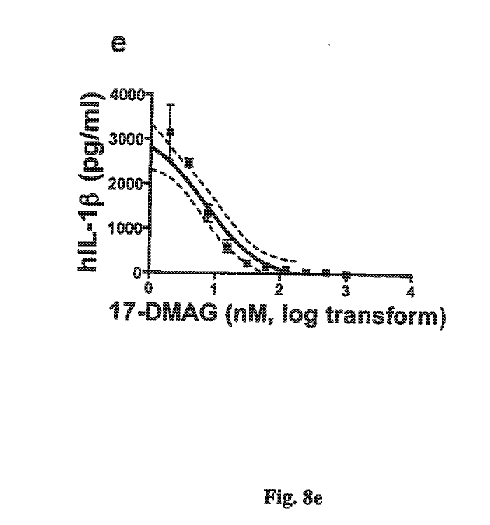

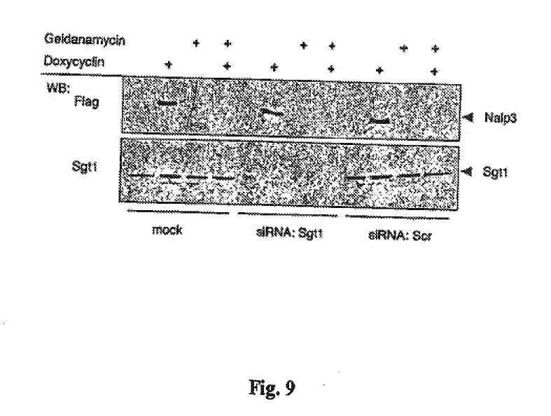

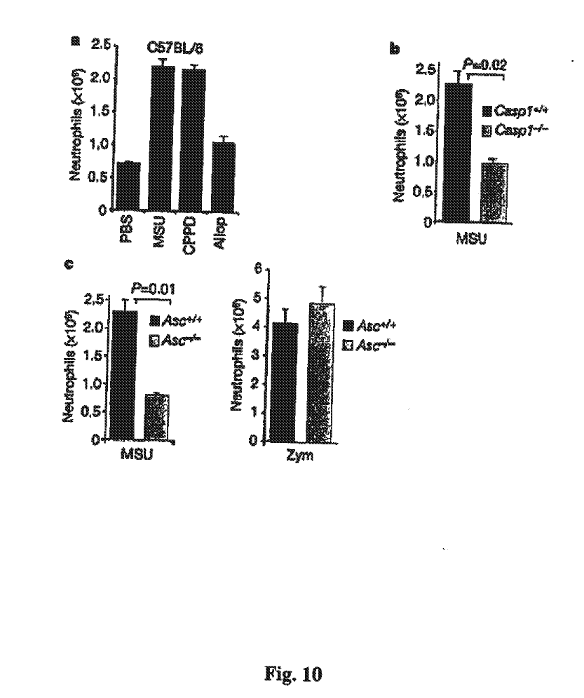

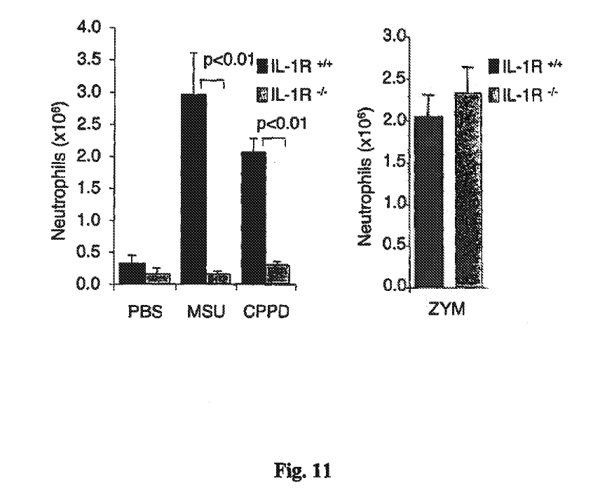

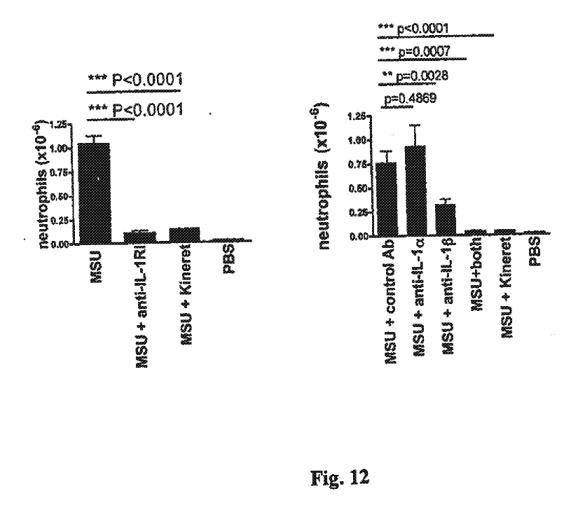

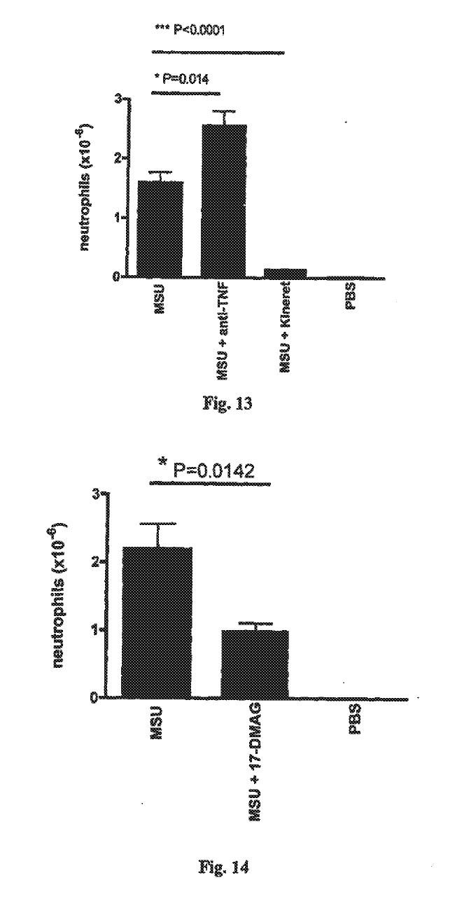

This example describes the production of mature IL-1β by a monocytic cell line of human origin (THP1) cells and by human monocytes in response to MSU or CPPD crystals. THP1 cells were incubated with MSU crystals and maturation of IL-1β was indeed detected following stimulation with as little as 10 μg/ml of the crystals (FIG. 1a). Addition of zYVAD-fmk, a known inhibitor of caspase-1 activation, completely blocked MSU-induced IL-1β activation, suggesting the dependency of proIL-1β cleavage on caspase-1 (FIG. 1a). CPPD, another type of pathogenic crystal involved in calcium pyrophosphate deposition disease, also known as pseudogout, was as active as MSU (FIG. 1b). Crystal-induced IL-1β processing was specific for these structures, as the non-inflammatory allopurinol or diamond crystals and particulate elements, such as zymosan and aluminum powder, failed to induce proIL-1β processing (FIG. 1c), despite their similar size and/or chemical composition. Compared to the known activators of the inflammasome, crude LPS and ATP, MSU and CPPD were more active^{11, 13} (FIG. 1c). This superior potency was particularly evident when analyzing processing of proIL-18, the second known substrate of caspase-1 (FIG. 1c). Previously, we demonstrated that the inflammatory caspases are cleaved and released along with active IL-1β following activation of the inflammasome¹³. This was also observed when cells were treated with MSU and CPPD (FIG. 1c, d). In order to exclude that crystal-mediated activation of caspase-1 is a unique property of the THP1 cell line only, MSU and CPPD were added to purified human monocytes. As shown in FIG. 1d, a strong response to both pathogenic crystals was also elicited in primary cells.

### Example 2

This example illustrates the direct involvement of the NALP3 inflammasome in crystal-induced inflammation. We analyzed peritoneal macrophages (PMΦs) derived from mice deficient in various key proteins of the inflammasome complex or other proinflammatory pathways. Given the absence and/or rapid degradation of proIL-1β in PMΦs ex vivo, and since we failed to see any direct induction of the transcription or translation of proIL-1β by MSU or CPPD, we stimulated the TLR4 in PMΦs with highly pure LPS to induce the synthesis of the cytokine^{11, 13}. Consistent with our previous findings in human monocytes, mouse PMΦs stimulated with MSU or CPPD activated caspase-1 and secreted mature IL-1β (FIG. 2a). Maturation was abolished in PMΦs from caspase-1 deficient mice, confirming the specificity of the activation. As expected, MyD88 deficient PMΦs did not produce mature IL-1β due to their defective TLR signaling, resulting in a failure to produce proIL-1β following LPS prestimulation (FIG. 2a). Nevertheless, MyD88−/− PMΦs still activated caspase-1 (FIG. 2a), further suggesting that this activation is TLR-independent and is consistent with a possible involvement of the inflammasome^{13, 15}. ASC is a crucial adaptor protein required for the recruitment of caspase-1 to the NALP inflammasome complex¹². ASC deficient PMΦs did not produce any mature IL-1β following stimulation by MSU and CPPD crystals (FIG. 2b).

The human genome harbors a repertoire of fourteen NALPs. It is currently not clear how many of them form inflammasomes. NALP3 is expressed in both monocytes and macrophages and is well conserved in human and mouse. We considered that the NALP3 inflammasome was possibly implicated in crystal-induced caspase-1 activation and we therefore generated NALP3 deficient mice (FIG. 3). Similar to PMΦs from ASC−/− mice, IL-1β release was impaired in NALP3-deficient PMΦs upon MSU and CPPD exposure (FIG. 2c). Likewise IL-1α release was impaired in NALP3-deficient mouse macrophages upon MSU exposure, but TNFα was not (FIG. 2d). IL-1β induction by ATP, the other known non-microbial stimulus of inflammasomes was also dependent on NALP3 (FIG. 2c). While blocking of the ATP-receptor P2X₇ inhibited ATP-driven inflammasome activation, it had no effect on MSU-induced activation, indicating that the two inflammasome-activating pathways act independently (FIG. 4).

### Example 3

This example illustrates that TNF production is dependent upon IL-1 secretion, in response to MSU or CPPD crystals. In addition to cytokines whose activity is dependent on caspase-1 activation, MSU and CPPD are known to induce other cytokines such as TNF-α^{17, 18}, suggesting additional, inflammasome-independent activities of the crystals. When assaying the release of TNF-α, we realized that the production of TNF-α was relatively slow and was preceded by the release of IL-1β¹⁹ (FIG. 5a). It was therefore possible that TNF-α secretion was initiated, at least in part, by the released mature IL-1β. Indeed, blocking the maturation of IL-1β with zYVAD-fmk reduced by more than 50% the production of TNF-α induced by MSU and CPPD, without affecting TNF-α production by the TLR2 agonist Zymosan (FIG. 5a). Similarly IL-1Ra, a natural inhibitor of IL-1 signaling, significantly affected TNF-α and IL-6 production by human monocytes (FIG. 5b).

### Example 4

This example illustrates that an intact cytoskeleton is required for IL-1β release upon challenge with MSU or CPPD crystals, and that colchicine (a drug preventing proper assembly of microtubules) inhibits NALP3 inflammasome activation. Pretreatment with intravenous colchicine prior to intraarticular MSU injections greatly reduces inflammation²³, suggesting that colchicine targets the initial phase of inflammation. We therefore investigated the role of colchicine in crystal-induced maturation of IL-1β. As shown in FIG. 6, pretreatment with colchicine, but not its solvent ethanol, completely blocked the processing of IL-1β. In contrast, colchicine did not affect IL-1β activation by extracellular ATP, indicating that the drug acts upstream of inflammasome activation.

### Example 5

This example describes small molecular compounds inhibiting the formation of NALP3 inflammasome for the treatment of gout or pseudogout. In many instances, assembly of multi-protein structures relies in the formation of complexes between individual components of the final protein multimer and chaperone proteins, such as heat shock proteins. It is also known from work conducted in plants that the activity of the plant disease resistance proteins (R proteins), which are structurally related to NALP proteins, is controlled by the HSP90 client protein Sgt1. In this experiment, it was observed that the production of mature IL-1β by human monocytes stimulated with monosodium urate (MSU) crystals was abrogated by the addition of geldanamycin, a known HSP90 inhibitor, in the culture medium. Geldanamycin also inhibited production of IL-1β induced by other stimuli, such as PGN and ATP (FIG. 7). Similarly, 17-AAG, a less toxic derivate of geldanamycin, almost completely inhibited production of IL-1β by mouse macrophages stimulated by ATP (FIG. 8b) or by MSU crystals (FIG. 8c), whereas TNFα production was reduced by less than 50% (FIG. 8a). Likewise, 17-DMAG, a water soluble derivate of geldanamycin, inhibited production of IL-1β by mouse macrophages (FIG. 8d) or human blood monocytes (FIG. 8e) stimulated by MSU crystals.

The loss of mature IL-1β secretion by geldanamycin could be due to an inhibitory effect at the level of IL-1 transcription, assembly of the inflammasome, activity of the inflammasome or secretion of mature IL-1β. In order to elucidate how geldanamycin prevents mature IL-1β secretion by MSU-stimulated monocytes (see previous FIG. 7), it was investigated whether NALP3 expression was impaired following geldanamycin treatment. It was observed that NALP3 expression was independent of the presence of Sgt1, since when Sgt1 expression was abrogated by Sgt1-specific siRNA treatment, expression of NALP3 was maintained. Upon addition of geldanamycin in the culture medium, NALP3 expression was abrogated, regardless of the presence or the absence of Sgt1 (FIG. 9). These results explain the inhibitory effect of geldanamycin on mature IL-1β secretion.

Together, these results illustrate the effect of geldanamycin, or its derivates 17-AAG and 17-DMAG, on the assembly of the NALP3 inflammasome, and thereby on the secretion of mature IL-1β. These results support the notion that molecules inhibiting formation of NALP3 inflammasome may be used in the treatment of gout or pseudogout.

### Example 6

This example describes the reduced in vivo inflammatory infiltrate in mice deficient for inflammasome components in response to MSU crystals. Clinically, gout and pseudogout are associated with edema and erythema of the joints, with consequent severe pain, conditions that are associated with strong infiltration of neutrophils in the intraarticular and periarticular spaces. This marked neutrophil influx can be reproduced experimentally in mice by intraperitoneal injection of crystals²⁴. We used this well-established model to investigate the in vivo role of the inflammasome in crystal-induced inflammation. MSU, CPPD or allopurinol crystals were injected and the peritoneal recruitment of neutrophils was analyzed 6 h later. Both MSU and CPPD elicited a considerable increase in the recruitment of neutrophils compared with PBS or allopurinol when injected in wild-type C57BL/6 mice (FIG. 10a). Importantly, when pathogenic crystals were injected in mice deficient in caspase-1 or ASC, neutrophil influx reduced by 60% (FIGS. 10b and c), indicating a main role of the inflammasome and IL-1β in this process. Of note, zymosan-induced neutrophil influx was not affected in ASC deficient mice (FIG. 10c).

### Example 7

This example illustrates that IL-1R engagement/triggering plays a determining role in gout symptoms in a mouse model. Aseptic peritonitis was induced by injection of MSU crystals in the peritoneal cavity of WT or IL-1RI deficient mice, or in combination with blocking monoclonal antibodies directed against the IL-1RI or with IL-1Ra (anakinra, Kineret) or in combination with blocking monoclonal antibodies directed against IL-1α and IL-1β. The recruitment of inflammatory cells was measured 6 h after injection. The IL-1R deficient mice exhibited a completely reduced recruitment of neutrophils following MSU and CPPD (FIG. 11). In the same line of evidence, administration of Kineret or monoclonal antibodies to IL-1RI or monoclonal antibodies to IL-1α and IL-1β almost entirely prevented neutrophil influx (FIG. 12). In contrast, zymosan-induced neutrophil influx was not affected in IL-1R deficient mice (FIG. 11).

### Example 8

This example describes that blocking TNFα does not alleviate clinical symptoms of gout in a mouse model. Aseptic peritonitis was induced by injection of MSU crystals in the peritoneal cavity of mice with or without blocking monoclonal antibodies directed against TNFα or with IL-Ra (anakinra, Kineret). The recruitment of inflammatory cells was measured 6 h after injection. As demonstrated on FIG. 13, administration of blocking monoclonal antibodies to TNFα did not reduce neutrophil influx, whereas Kineret did.

### Example 9

This example describes that administration of 17-DMAG, a water-soluble and orally available HSP90 inhibitor, does alleviate clinical symptoms of gout in a mouse model. Aseptic peritonitis was induced by injection of MSU crystals in the peritoneal cavity of BALB/C mice, alone or in combination with 25 mg/kg 17-DMAG. The recruitment of inflammatory cells was measured 6 h after injection. As shown on FIG. 14, administration of 17-DMAG reduced neutrophil influx in response to MSU.

### Example 10

This example describes that inhibition of IL-1 in patients with acute gout ameliorates clinical symptoms of gout. Four patients with proven gout were treated with anakinra (Kineret).

### Case 1

A 72 year-old woman with a 13-year history of chronic tophaceous gout was evaluated for treatment by recombinant uricase. Previously, she had a severe cutaneous reaction to allopurinol, and uricosuric treatment with benzbromazone caused renal stones. During previous gout flares, medical treatment was unsatisfactory. She could only tolerate a low dose of diclofenac (50-100 mg/d), as higher doses caused gastrointestinal side effects including one episode of G-I hemorrhage. Colchicine at 1 mg per day provoked intolerable diarrhea, and oral corticosteroid caused severe abdominal pain. Uricase treatment was commenced (14 mg iv daily for 5 days) and resulted in rapid lowering of her uric acid levels. On the 4^{th} day of treatment, arthritis developed in her hand and foot joints. As treatment with diclofenac during previous flares took more than a week to relieve her symptoms, treatment with anankinra was proposed. Anakinra was administered at 100 mg daily for 2 days. Her arthritis responded rapidly, and there was a rapid reduction in joint pain over 48 h. She was able to continue the course of uricase. No acute flares were observed during the following 2 months.

### Case 2

A 70 year-old man with an 8-year history of chronic tophaceous gout was assessed for hypouricemic treatment. Past medical history included congestive cardiac failure, severe ischemic heart disease, hypertension and renal insufficiency (serum creatinine of 202 μmol/L, normal range 44-80 μmol/L). Previous trials of treatment with allopurinol had to be abandoned because acute gout developed after the first dose, which did not respond to small doses of NSAIDs. Higher doses of NSAIDs were contraindicated because of renal failure. Colchicine at low doses (<1 mg/d) provoked rapid onset of diarrhea. The patient was started again on a low dose of allopurinol (100 mg), and after the first dose, developed acute arthritis of the right foot and ankle. Anankinra 100 mg daily was administered for 3 days with rapid resolution of signs and symptoms of arthritis. He continued on allopurinol 100 mg daily and on follow-up one month later, had no further flare-ups while continuing on the same dose of allopurinol.

### Case 3

A 72 year-old man with a past history of diabetes, hypertension, renal failure and ischemic heart disease presented with polyarticular gout. Arthritis involved the knees, the right ankle and the right tarsal joints. MSU crystals were detected in the knee joint aspirate and the fluid was inflammatory (leucocyte count 22.5 G/L, 98% polymorphonuclear). Because of renal impairment and a history of rectal bleeding on colchicine, treatment with oral prednisone at 30 mg daily was started with a tapering dose over 7 days. Despite steroids, the arthritis remained active and the patient could not walk. After steroids were stopped, the patient received a 3-day course of anakinra, with complete resolution of arthritis by the second day. On follow up one month later, there had been no recurrence of arthritis.

### Case 4

A 50 year-old man with a 20-year history of polyarticular gout principally involving the right ankle and big toe seeked medical advice because of increasingly frequent gout attacks. He had started allopurinol but could not continue treatment because it induced flare-ups of arthritis. NSAIDs caused severe gastrointestinal pain and colchicine at 1 mg daily was ineffective. Higher doses of colchicine provoked diarrhea. On examination, he had arthritis of the right ankle joint and MSU crystals were identified in the joint aspirate. A trial of anakinra was initiated at the same time as starting allopurinol (300 mg/d). The patient's arthritis responded rapidly and he continued on allopurinol. He was asymptomatic two months later.

### REFERENCES

1. Wollaston, H. W. On Gouty and Urinary Concretions. Phil. Trans. 87, 386-400 (1797).
2. Shi, Y., Evans, J. E. & Rock, K. L. Molecular identification of a danger signal that alerts the immune system to dying cells. Nature 425, 516-21 (2003).
3. Galon, J., Aksentijevich, I., McDermott, M. F., O'Shea, J. J. & Kastner, D. L. TNF-α RSF1A mutations and autoinflammatory syndromes. Curr Opin Immunol 12, 479-86 (2000).
4. Martinon, F. & Tschopp, J. Inflammatory caspases: linking an intracellular innate immune system to autoinflammatory diseases. Cell 117, 561-74 (2004).
5. Stojanov, S. & Kastner, D. L. Familial autoinflammatory diseases: genetics, pathogenesis and treatment. Curr Opin Rheumatol 17, 586-99 (2005).
6. Dinarello, C. A. Blocking IL-1 in systemic inflammation. J Exp Med 201, 1355-9 (2005).
7. Shoham, N. G. et al. Pyrin binds the PSTPIP1/CD2BP1 protein, defining familial Mediterranean fever and PAPA syndrome as disorders in the same pathway. Proc Natl Acad Sci USA 100, 13501-6 (2003).
8. Chae, J. J. et al. Targeted disruption of pyrin, the FMF protein, causes heightened sensitivity to endotoxin and a defect in macrophage apoptosis. Mol Cell 11, 591-604 (2003).
9. Burns, K., Martinon, F. & Tschopp, J. New insights into the mechanism of IL-1beta maturation. Curr Opin Immunol 15, 26-30 (2003).
10. Martinon, F., Burns, K. & Tschopp, J. The inflammasome: a molecular platform triggering activation of inflammatory caspases and processing of proIL-beta. Mol Cell 10, 417-26. (2002).
11. Mariathasan, S. et al. Differential activation of the inflammasome by caspase-1 adaptors ASC and Ipaf. Nature 430, 213-8 (2004).
12. Tschopp, J., Martinon, F. & Burns, K. NALPs: a novel protein family involved in inflammation. Nat Rev Mol Cell Biol 4, 95-104 (2003).
13. Martinon, F., Agostini, L., Meylan, E. & Tschopp, J. Identification of bacterial muramyl dipeptide as activator of the NALP3/cryopyrin inflammasome. Curr Biol 14, 1929-34 (2004).
14. Martinon, F. & Tschopp, J. NLRs join TLRs as innate sensors of pathogens. Trends Immunol (2005).
15. Yamamoto, M. et al. ASC is essential for LPS-induced activation of procaspase-1 independently of TLR-associated signal adaptor molecules. Genes Cells 9, 1055-67 (2004).
16. Faires, J. S. & McCarty, D. J. Acute Arthritis in Man and Dog after Intrasynovial Infection of Sodium Urate Crystals. Lancet 280, 682-685 (1962).
17. Dalbeth, N. & Haskard, D. O. Mechanisms of inflammation in gout. Rheumatology (Oxford) 44, 1090-6 (2005).
18. Meng, Z. H., Hudson, A. P., Schumacher, H. R., Jr., Baker, J. F. & Baker, D. G. Monosodium urate, hydroxyapatite, and calcium pyrophosphate crystals induce tumor necrosis factor-alpha expression in a mononuclear cell line. J Rheumatol 24, 2385-8 (1997).
19. Chapman, P. T. et al. Endothelial activation in monosodium urate monohydrate crystal-induced inflammation: in vitro and in vivo studies on the roles of tumor necrosis factor alpha and interleukin-1. Arthritis Rheum 40, 955-65 (1997).
20. Hoffman, H. M. et al. Prevention of cold-associated acute inflammation in familial cold autoinflammatory syndrome by interleukin-1 receptor antagonist. Lancet 364, 1779-85 (2004).
21. Hawkins, P. N., Lachmann, H. J. & McDermott, M. F. Interleukin-1-receptor antagonist in the Muckle-Wells syndrome. N Engl J Med 348, 2583-4 (2003).
22. Molad, Y. Update on colchicine and its mechanism of action. Curr Rheumatol Rep 4, 252-6 (2002).
23. Malawista, S. E. & Seegmiller, J. E. The Effect of Pretreatment with Colchicine on the Inflammatory Response to Microcrystalline Urate: A Model for Gouty Inflammation. Ann Intern Med 62, 648-57 (1965).
24. Getting, S. J. et al. Molecular determinants of monosodium urate crystal-induced murine peritonitis: a role for endogenous mast cells and a distinct requirement for endothelial-derived selectins. J Pharmacol Exp Ther 283, 123-30 (1997).
25. Goldfinger, S. E., Howell, R. R. & Seegmiller, J. E. Suppression of metabolic accompaniments of phagocytosis by colchicine. Arthritis Rheum 8, 1112-22 (1965).
26. Liu-Bryan, R., Scott, P., Sydlaske, A., Rose, D. M. & Terkeltaub, R. Innate immunity conferred by Toll-like receptors 2 and 4 and myeloid differentiation factor 88 expression is pivotal to monosodium urate monohydrate crystal-induced inflammation. Arthritis Rheum 52, 2936-46 (2005).
27. Janeway, C. A., Jr. & Medzhitov, R. Innate immune recognition. Annu Rev Immunol 20, 197-216 (2002).
28. Matzinger, P. The danger model: a renewed sense of self. Science 296, 301-5. (2002).
29. Hoffman, H. M., Mueller, J. L., Broide, D. H., Wanderer, A. A. & Kolodner, R. D. Mutation of a new gene encoding a putative pyrin-like protein causes familial cold autoinflammatory syndrome and Muckle-Wells syndrome. Nat Genet 29, 301-5. (2001).
30. Agostini, L. et al. NALP3 forms an IL-1beta-processing inflammasome with increased activity in Muckle-Wells autoinflammatory disorder. Immunity 20, 319-25 (2004).
31. Schiltz, C. et al. Monosodium urate monohydrate crystal-induced inflammation in vivo: quantitative histomorphometric analysis of cellular events. Arthritis Rheum 46, 1643-50 (2002).
32. Pouliot, M., James, M. J., McColl, S. R., Naccache, P. H. & Cleland, L. G. Monosodium urate microcrystals induce cyclooxygenase-2 in human monocytes. Blood 91, 1769-76 (1998).
33. Tsuchiya, S. et al. Induction of maturation in cultured human monocytic leukemia cells by a phorbol diester. Cancer Res 42, 1530-6. (1982).

1. Use of an inhibitor blocking IL-1 or its maturation by the NALP3 inflammasome for the preparation of a medicament used in the treatment of gout or pseudogout.

2. The use of claim 1, characterized in that IL-1 is selected among the group consisting in IL-1α and IL-1β.

3. The use of claim 1 or 2, characterized in that the inhibitor blocking IL-1 maturation by the NALP3 inflammasome is selected among the group consisting of inhibitors of NALP3 inflammasome formation and inhibitors of NALP3 inflammasome activity.

4. The use of claim 3, characterized in that the inhibitor of NALP3 inflammasome formation is an inhibitor of HSP90.

5. The use of claim 4, characterized in that the inhibitor of HSP90 is selected among the group consisting in geldanamycin, 17-AAG (17-(Allylamino)-17-demethoxygeldanamycin) and 17-DMAG (17-(Dimethylaminoethylamino)-17-demethoxygeldanamycin).

6. The use of claim 1 or 2, characterized in that the inhibitor blocking IL-1 is selected among antibodies inhibiting activity of IL-1.

7. The use of claim 6, characterized in that the anti-IL-1 antibody is a monoclonal antibody.

8. The use of claim 1 or 2, characterized in that the inhibitor blocking IL-1 is selected among antibodies inhibiting binding of IL-1 to its receptor (IL-1R type I).

9. The use of claim 8, characterized in that the antibody is an anti-IL-1RI antibody.

10. The use of claim 9, characterized in that the anti-IL-1RI antibody is a monoclonal antibody.

11. The use of claim 1 or 2, characterized in that the inhibitor blocking IL-1 is an IL-1 receptor antagonist (IL-1Ra).

12. The use of claim 11, characterized in that the IL-1Ra is anakinra.

13. The use of one of claims 1 to 12, characterized in that the inhibitor blocking IL-1 or its maturation by the NALP3 inflammasome is administered once a day, in a dose sufficient to inhibit IL-1 activity.

14. The use of one of claims 1 to 12, characterized in that the inhibitor blocking IL-1 or its maturation by the NALP3 inflammasome is administered once a week, in a dose sufficient to inhibit IL-1 activity.

15. The use of one of claims 1 to 12, characterized in that the inhibitor blocking IL-1 or its maturation by the NALP3 inflammasome is administered once a month, in a dose sufficient to inhibit IL-1 activity.

16. The use of claim 12, characterized in that anakinra is administered once a day at a dose of 100 mg/day for 1, 2 or 3 days.

17. The use of one of claims 1 to 16, characterized in that the inhibitor blocking IL-1 or its maturation by the NALP3 inflammasome is used in combination with at least a second anti-gout compound.

18. The use of claim 17, characterized in that the second anti-gout compound is selected among the group consisting of colchicines, allopurinol and uricase.

19. The use of one of claims 1 to 18, characterized in that the inhibitor blocking IL-1 or its maturation by the NALP3 inflammasome is used in combination with at least an anti-inflammatory compound or composition.

20. The use of claim 19, characterized in that the anti-inflammatory compound or composition is selected among the group consisting of prednisone, betamethasone, dexamethasone, methylprednisolone, prednisolone, cortivazol, hydrocortisone, triamcinolone, indimetacine, sulindac, tiaprofenic acid, alminoprofene, diclofenac, etodolac, flurbiprofene, ibuprofene, ketoprofene, nabumetone, naproxene, meloxicam, piroxicam, tenoxicam, celecoxib and refecoxib.

21. Use of an inhibitor of HSP90 for the preparation of a medicament used in the treatment of inflammatory disorders.

22. The use of claim 21, characterized in that the inhibitor of HSP90 is selected among the group consisting in geldanamycin, 17-AAG (17-(Allylamino)-17-demethoxygeldanamycin) and 17-DMAG (17-(Dimethylaminoethylamino)-17-demethoxygeldanamycin).

23. The use of claim 21 or 22, characterized in that the inflammatory disorders are selected among the group consisting of autoimmune and auto-inflammatory diseases, such as gout, pseudogout, Muckle-Wells Syndrome, hereditary periodic fevers, Familial Mediterranean Fever, Familial Cold-induced Autoinflammatory Syndrome, Blau Syndrome, rheumatoid arthritis, osteoarthritis, systemic-onset juvenile idiopathic arthritis, psoriasis, lupus, multiple sclerosis, asthma, chronic obstructive pulmonary disorder, inflammatory bowel disease, Crohn's disease, atherosclerosis, Alzheimer's disease, Parkinson's disease.
